(19) United States
(12) Patent Application Publication
(10) Pub. No.: US 2022/0359088 A1
(43) Pub. Date: Nov. 10, 2022

(54) GRAPHICAL USER INTERFACES AND PROCESSES FOR ADVANCED SEARCHING OF DATABASES FOR RELEVANT DATA VALUES

(71) Applicant: University of Rochester, Rochester, NY (US)

(72) Inventors: Kostantinos Vasalos, Webster, NY (US); Kathleen Fear, Rochester, NY (US)

(21) Appl. No.: 17/734,522

(22) Filed: May 2, 2022

Related U.S. Application Data
(60) Provisional application No. 63/183,777, filed on May 4, 2021.

(51) Int. Cl. G16H 50/70; G06F 16/2457; G16H 10/60; G06F 16/242; G16H 50/20

(52) U.S. Cl. CPC G16H 50/70; G06F 16/24578; G16H 10/60; G06F 16/2423; G16H 50/20

(57) ABSTRACT

Computer databases can be searched for relevant data values using an advanced search process. For example, a system can receive a search query indicating an attribute and a condition of a target client. The search query can be for searching one or more databases for data values relevant to the target client. The system can determine other clients described in the databases that are similar to the target client based on the attribute. The system can then filter the other clients to determine which of them has been treated for the condition. The system can identify candidate providers that treated said clients for the condition based on relationships in the databases. The system can then generate an ordered list of the candidate providers based on corresponding treatment metrics and transmit the ordered list of the candidate providers as search results in response to the search query.

### CROSS-REFERENCE TO RELATED APPLICATION

This application claims the benefit of and priority to U.S. Provisional Application No. 63/183,777, filed May 4, 2021, which is incorporated herein by reference in its entirety.

### TECHNICAL FIELD

The present disclosure relates generally to searching information in computer databases. More specifically, but not by way of limitation, this disclosure relates to graphical user interfaces and processes for searching through largescale databases for relevant data values in response to search queries from client devices.

### BACKGROUND

Computer databases have become increasingly large and complex, often storing millions or tens of millions of data entries. Analyzing such databases has also become increasingly complex. As a result, it can be challenging for software developers to write software that can quickly and efficiently search through one or many databases to identify relationships between relevant data values. Such software often implements suboptimal algorithms that consume considerable amounts of computing resources (e.g., CPU, RAM, and storage) to process and analyze databases information, which can impact the performance of other software that would otherwise consume those computing resources. The searching process can become even more cumbersome and computationally expensive when the relationships sought are more subjective and based on multiple factors. In such cases, search queries can become exceedingly complex and many iterations of searching may need to be performed to obtain suitable results, further exhausting computing resources.

### BRIEF DESCRIPTION OF THE DRAWINGS

FIG. 1 shows a block diagram of an example of a system according to some aspects of the present disclosure.

FIG. 2 shows a flow chart of an example of a search process according to some aspects of the present disclosure.

FIG. 3 shows an example of a database architecture according to some aspects of the present disclosure.

FIG. 4 shows an example of a graphical user interface according to some aspects of the present disclosure.

FIGS. 5-14 show examples of pages in the graphical user interface according to some aspects of the present disclosure.

### DETAILED DESCRIPTION

Certain aspects and features of the present disclosure involve a graphical user interface and corresponding process to search through one or more computer databases, so as to identify and rank relevant data values in a way that can be faster and more accurate than conventional approaches. The graphical user interface and search process may be particularly well suited to queries that are more complex and multi-factored, such as if attempting to match service providers to clients based on attributes of the service providers and the attributes of the clients, though aspects of the present disclosure can also be used in other contexts. Conventional searching software may consume considerable amounts of computing resources and provide suboptimal results for these types of searches, while some examples of the present disclosure may produce better results in a more computationally efficient way.

As one particular example, a system of the present disclosure can be used in the context of a client searching for a medical provider to seek treatment for a physical condition. In this example, the system can provide a graphical user interface that has multiple fields through which a user can input multiple attributes of the client, where the client may or may not be the same as the user of the system. For example, the user of the system may be office personnel aiding the client. The graphical user interface can also include a field through which the user can input the condition for which treatment is sought. The user can input this information and transmit the search query to the system, which can respond to the search query by executing an advanced searching process to identify and rank treatment providers and present the results in the graphical user interface. The advanced searching process can be implemented as follows.

The system can receive the search query and responsively determine similarity values corresponding to other clients described in one or more databases. Each similarity value can indicate a respective similarity between the target client and one of the other clients described in the database. In some examples, the system can determine a similarity value between the target client and another client by executing a machine-learning model, such as a neural network or a classifier, or another type of model. Additionally or alternatively, the system can determine a similarity value between the target client and another client by counting the number of overlapping attributes that are present between the target client and the other client. Any suitable process or processes can be used to determine the similarity values.

After determining the similarity values, the system can select a subset of the other clients from the one or more databases by comparing their similarity values to a predefined similarity threshold. For example, the system can select a subset of the other clients that have corresponding similarity values that exceed the predefined similarity threshold. An example of the similarity threshold may be 75 for a range of similarity values spanning from 0 to 100. This step of the search process may help ensure that subsequent steps are performed using data about other clients that are most similar to the target client, which can yield more accurate results.

Next, the system can filter the subset of other clients to determine which of the other clients are or were affected by the condition for which treatment is sought. Such clients can be referred to as affected clients. For example, the system can query the one or more databases to determine which, if any, of the other clients in the subset are marked as having been treated for the condition. In this way, the searching process can enable the system to determine which of the other similar clients have also been treated for the same condition.

Having identified other similar clients that have been treated for the same condition, the system can identify one or more providers that treated the other affected clients. Since such information can be stored in the one or more databases, the system can determine this information based on relationships between the affected clients and the providers in the one or more databases. These providers can serve as candidates for treating the target client, so they can be referred to as candidate providers.

The system may next order the candidate providers relative to one another based on treatment metrics corresponding to the candidate providers. For example, the system can determine a value (e.g., a score) for each candidate provider based on one or more treatment metrics and compare the values to one another to order the candidate providers. The treatment metrics can quantify one or more aspects of a treatment program for the condition that was implemented by the candidate provider. After ordering the candidate providers, the system can provide the ordered list of candidate providers back to the user via the graphical user interface. The user may then select a provider from the list to treat the target client's condition.

Using the graphical user interface and corresponding search process described above, the user experience and search accuracy can be improved. The graphical user interface can allow a user to easily craft a search query using human-readable and digestible terms (e.g., rather than using a complex string of advanced search operators) to describe the attributes and the condition of the target client. Upon receiving a search query from a user via the graphical user interface, the system can then apply the advanced search process to the search query, so as to identify and produce an ordered list of providers that have treated clients that are similar to the target client in a way that yielded desirable treatment metrics. These search results can be more optimal than alternative approaches that fail to consider the attributes of the target client and/or the treatment metrics. Additionally, the advanced searching process can involve multiple stages of filtering (e.g., identifying other similar clients and then filtering the other similar clients down to those that had the same condition), each of which can significantly refine the search space to reduce the amount of computer resources that are consumed in performing later stages of the searching process. By continually narrowing the search space in this way, the volume of data to be searched and the computational overhead associated with executing the search process on a computer can be reduced.

While the above examples are provided in a particular context, it will be appreciated that similar concepts can be applied to other contexts. The providers may be any suitable type of service provider that can provide any suitable type of service to a client (e.g., an individual or entity) in relation to any suitable condition.

These illustrative examples are given to introduce the reader to the general subject matter discussed here and are not intended to limit the scope of the disclosed concepts. The following sections describe various additional features and examples with reference to the drawings in which similar numerals indicate similar elements but, like the illustrative examples, should not be used to limit the present disclosure.

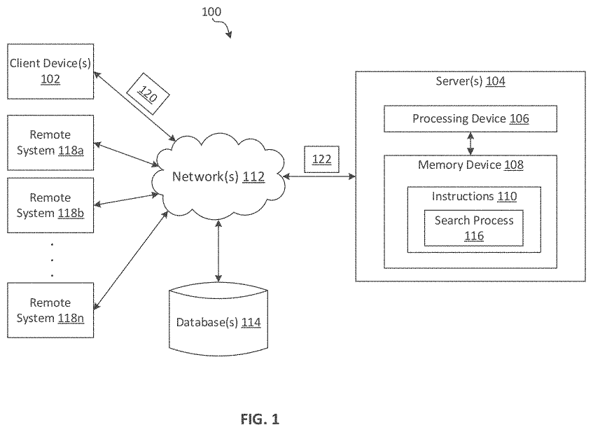

FIG. 1 shows a block diagram of an example of a system 100 according to some aspects of the present disclosure. The system 100 includes one or more client devices 102, such as laptop computers, desktop computers, mobile phones, or another type of computing devices. The client devices 102 are in communication with one or more servers 104 via one or more networks 112, such as a local area network or the Internet. The servers 104 can generate a graphical user interface through which users of the client devices 102 can generate and submit search queries 120 to the servers 104. The servers 104 can receive the search queries 120, execute a search process 116 for searching one or more databases 114 based on the search queries 120, and provide search results back to the client devices 102. This search process is described in greater detail later on.

As shown, the servers 104 include a processing device 106 communicatively coupled to a memory device 108. Although the processing device 106 and the memory device 108 are shown as part of the same computing device in FIG. 1, in other examples they can be distributed from (e.g., remote to) one another. The processing device 106 can include one processor or multiple processors. Non-limiting examples of the processing device 106 include a Field-Programmable Gate Array (FPGA), an application-specific integrated circuit (ASIC), a microprocessor, etc. The processing device 106 can execute instructions 110 stored in the memory device 108 to perform operations. The instructions 110 may include processor-specific instructions generated by a compiler or an interpreter from code written in any suitable computer-programming language, such as C, C++, C#, Python, or Java.

The memory device 108 can include one memory or multiple memories. The memory device 108 can be non-volatile and may include any type of memory that retains stored information when powered off. Non-limiting examples of the memory device 108 include electrically erasable and programmable read-only memory (EEPROM), flash memory, or any other type of non-volatile memory. At least some of the memory device 108 can include a non-transitory computer-readable medium from which the processing device 106 can read instructions 110. A computer-readable medium can include electronic, optical, magnetic, or other storage devices capable of providing the processing device 106 with computer-readable instructions or other program code. Non-limiting examples of a computer-readable medium include magnetic disk(s), memory chip(s), ROM, random-access memory (RAM), an ASIC, a configured processor, optical storage, or any other medium from which a computer processor can read the instructions 110.

In some examples, the servers 104 can communicate with one or more remote systems 118a-n to generate the databases 114. The remote systems 118a-n may correspond to third parties that can collect information from the group of clients, the group of service providers, and/or other sources and transmit that information to the server 104. For example, the servers 104 can communicate with remote system 118a to receive attributes of a group of clients and store their attributes in the databases 114. Examples of the attributes can include demographic characteristics (DCs) such as sex, age, ethnicity, geographical location, or education level; socioeconomic characteristics of the target client; one or more aspects of the target client's medical history; preferences of the of the target client; or any combination of these. The servers 104 may also communicate with remote system 118b to receive attributes of a group of service providers and store their attributes in the databases 114. Examples of the attributes can include DCs of the service provider, a skill level of the service provider, a training level of the service provider, an area of expertise/specialization of the service provider, a communication style of the service provider, preferences of the service provider, an engagement level of the service provider, or any combination of these. The servers 104 may further communicate with remote system 118n to receive service data and store the treatment data in the databases 114. Examples of the service data can include outcomes and expenditures corresponding to one or more services provided by a service provider. For instance, the service data can be treatment data indicating treatment outcomes and expenditures relating to one or more treatment programs implemented by one or more treatment providers. The servers 104 may also interface with other remote systems to generate the databases 114. The servers 104 can receive, process, and aggregate any amount and combination of data from any number and combination of remote systems 118a-n to generate the databases 114.

The servers 104 can access and search the databases 114 to respond to search queries 120 from the client devices 102. In some examples, the servers 104 may execute multiple instances of the search process 116 concurrently (e.g., in parallel) to respond to multiple search queries 120 at faster speeds. For example, the servers 104 can execute multiple processing threads concurrently, where each processing thread can implement its own instance of the search process 116 to generate a search result for one of the search queries 120 and provide the search result in a response 122 to the client devices 102.

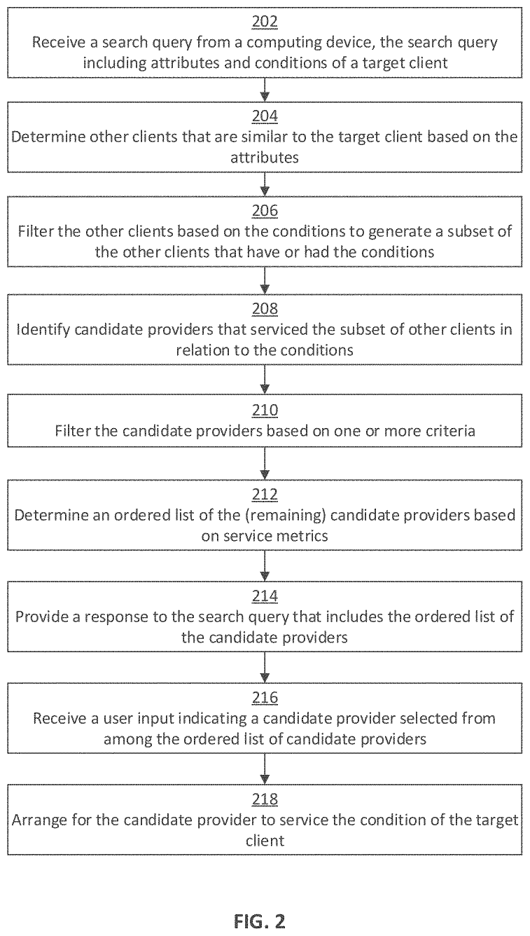

One example of this search process 116 is shown in FIG. 2. Of course, other examples may involve more operations, less operations, different operations, or a different order of the operations than is shown in FIG. 2. The search process 116 is described below with reference to the components of FIG. 1 described above.

In block 202, the server 104 receives a search query 120 from a computing device, such as client device 102. The search query 120 includes one or more attributes of a target client and one or more conditions of the target client. The target client can be an individual or entity that wants certain services. The target client can be the same as, or different from, the user of the computing device. The conditions can correspond to a physical state of the target client and can be different from the attributes.

In some examples, a user of the computing device can generate the search query 120 via a graphical user interface. The graphical user interface can have multiple fields for receiving input data (e.g., the one or more attributes and the one or more conditions) from the user. The graphical user interface can then generate and transmit the search query 120 based on the input data, for example in response to the selection of a submit button by the user.

In block 204, the server 104 determines other clients that are similar to the target client based on the one or more attributes, where the other clients are described in one or more databases 114. The other clients can be individuals or entities that have received services in the past. Information about the other clients and the services they received can be stored in the one or more databases 114. The server 104 can extract information about (e.g., attributes of) the other clients from the databases 114, determine similarity values for the other clients based on the extracted information, and determine that the other clients are similar to the target client based on the similarity values. Each similarity value can indicate a respective similarity of the target client to one of the other clients.

The server 104 can determine the similarity values for the other clients using any suitable approach or combination of approaches. For example, the server 104 can determine the similarity values by executing a machine-learning model or another type of model. The server 104 can provide a first set of attributes of the target client and a second set of attributes of the other client as input to the model. The model can analyze the first set of attributes and the second set of attributes to generate a similarity value for the other client. A higher similarity value may indicate that the other client is more similar to the target client, and a lower similarity value may indicate that the other client is less similar to the target client. As another example, the server 104 can apply a weighted algorithm to the first set of attributes and the second set of attributes to determine a relative similarity between the target client and the other client. Other approaches to determining the similarity values are also possible.

After determining the similarity values, the server 104 may compare the similarity values to a predefined similarity threshold to identify which of the other clients are sufficiently similar to the target client. For example, the server 104 can determine which of the other clients have corresponding similarity values that exceed the predefined similarity threshold. Those other clients can be considered similar to the target client.

In block 206, the server 104 filters the other clients (that are similar to the target client) based on the one or more conditions to generate a subset of the other clients that currently have, or that previously had, the one or more conditions. For example, the server 104 can access the databases 114 to determine historical information about conditions experienced by the other clients. The historical information can indicate which of the other clients currently have, or previously had, the one or more conditions. The server 104 can use the historical information to filter the other clients based on the one or more conditions, so as to generate a subset of the other clients associated with the one or more conditions.

In block 208, the server 104 identifies candidate providers that serviced (e.g., treated) the subset of the other clients in relation to the one or more conditions. For example, the databases 114 can include historical information about conditions experienced by the other clients and serviced by various providers. The server 104 can access this historical information to determine which candidate providers serviced the subset of the other clients in relation to the one or more conditions.

In block 210, the server 104 filters the candidate providers based on one or more criteria (e.g., to exclude candidate providers that do not satisfy the one or more criteria). In some examples, the criteria may be input by the user of the computing device and provided as part of the search query 120. Alternatively, the criteria may be input by the target client or predefined by an administrator of the system. Examples of the criteria can include the candidate provider having a certain amount of availability; the candidate provider having certain attributes; or any combination of these. Some or all of this information can be stored in the databases 114. The server 104 can access the databases 114 to determine which of the candidate providers do, and do not, satisfy the criteria.

In block 212, the server 104 determines an ordered list of the candidate providers (e.g., remaining after the filtering of block 210) based on one or more service metrics. One example of service metrics can be treatment metrics associated with treatments provided by the candidate providers to the other clients. In some such examples, the treatment metrics can include a success score indicating the success (or failure) of a certain treatment. Information about the success of a treatment may be gathered by a third party, for example by presenting the treated client with surveys at the start and end of treatment. Additionally or alternatively, the treatment metrics can include a value score indicating a cost associated with some or all of a treatment. In some examples, the treatment metrics can include a ratio of one type of treatment metric to another type of treatment metric. For example, the treatment metrics can include a treatment-value score generated by dividing a success score by a value score. Any other suitable treatment metrics may be used.

Additionally or alternatively to using the service metrics, the server 104 can determine the ordered list of the candidate providers based on the attributes of the providers. For example, the server 104 can determine the ordered list of the candidate providers based on one or more service metrics and one or more attributes of the providers. In one such example, the server 104 can determine a weighted rank for each provider in the ordered list by applying a first weight to the service metrics and a second weight to a particular attribute of the provider.

In block 214, the server 104 provides a response 122 to the search query 120 that includes the ordered list of the candidate providers. In some examples, the server 104 may output the ordered list in the graphical user interface to the user, so that the user can select a particular candidate provider from the ordered list of candidate providers.

In some examples, the server 104 may determine availability information for the candidate providers (in the ordered list). The availability information can specify times at which the candidate providers have open slots to meet with the target client within a predefined future timeframe, such as within the next 30 days. The server 104 can provide this availability information in the response 122 to aid the user in selecting a candidate provider that is suitably available to service the target client.

In block 216, the server 104 receives a user input indicating a candidate provider selected from among the ordered list of candidate providers. For example, the user of the computing device may provide user input to the graphical user interface to choose the candidate provider.

In block 218, the server 104 arranges for the candidate provider to service the condition of the target client. For example, the server 104 may generate an event (e.g., a meeting) on the schedules of the target client and/or the candidate provider so that the two can interact. Additionally or alternatively, the server 104 may designate the candidate provider to service the condition of the target client in the one or more databases 114.

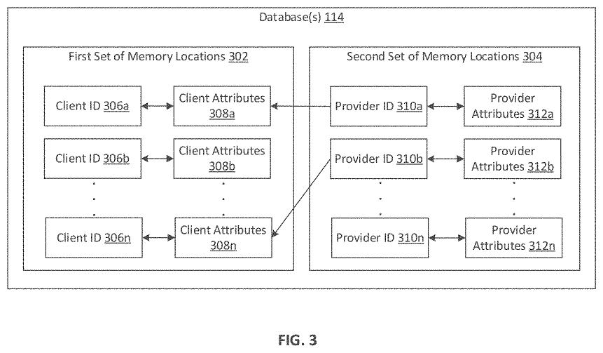

One example of a database 114 usable with the above search process 116 is shown in FIG. 3. In this example, the database 114 can include a first set of memory locations 302. The first set of memory locations 302 can include client identifiers 306a-n that identify clients. Examples of the client identifiers 306a-n can include names or unique client numbers. The client identifiers 306a-n can correspond to client attributes 308a-n. The client attributes 308a-n are attributes of the clients identified by the client identifiers 306a-n. For example, the client attributes 308a correspond to the client that is identified by the client identifier 306a.

The database 114 also includes a second set of memory locations 304. The second set of memory locations 302 can include provider identifiers 310a-n that identify providers. Examples of the provider identifiers 310a-n can include names or unique provider numbers. The provider identifiers 310a-n can correspond to provider attributes 312a-n. The provider attributes 312a-n are attributes of the providers identified by the provider identifiers 310a-n. For example, the provider attributes 312a correspond to the provider that is identified by the provider identifier 310a.

In some examples, the provider identifiers 310a-n can be included in the client attributes 308a-n. This is represented in FIG. 3 by dashed arrows between provider identifiers 310a, 310b and client attributes 308a, 308n. For example, the client attributes 308a can include a medical history of the corresponding client, where the medical history indicates that the client was treated for a particular condition by the provider corresponding to provider identifier 310a.

Using this database structure, the search process 116 can identify clients that are similar to a target client based on the client attributes 308a-n. The search process 116 can then determine which of the similar clients were treated for a target condition based on the client attributes 308a-n, and determine which of the providers treated the similar clients for the target condition based on the attributes 308a-n. The search process 116 can next determine attributes for those providers. Based on the attributes of the providers, the search process 116 can generate search results. The search results may include a list of candidate providers ordered based on their attributes.

It will be appreciated that the database 114 shown in FIG. 3 is intended to be illustrative and non-limiting. Other examples may include more information, less information, different information, or a different configuration of the information shown in FIG. 3.

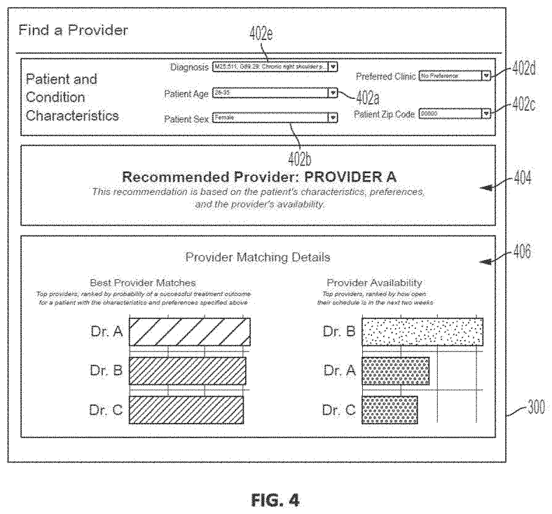

One example of a graphical user interface 400 designed to support the search process 116 described above is shown in FIG. 4. The graphical user interface 400 can be generated by the server 103 for output on a display of a computing device, such as the client device 102 of FIG. 1.

In this example, the graphical user interface 400 includes a set of input fields 402a-e. Input fields 402a-c are for receiving attributes of the target client, input field 402d is for receiving a preference of the target client relating to a service location, and input field 402e is for receiving a condition of the target client. Other examples may include more, less, or different input fields that are shown in FIG. 4. A user can input this information and press a button to generate and transmit a search query 120 to the server 104. The server 104 can receive the search query 120, execute a search process 116 for searching one or more databases 114 to determine ranked (e.g., ordered) list of candidate providers, and provide the ranked list of candidate providers to the computing device for display in frame 406 of the graphical user interface 400.

In some examples, the candidate providers can be ranked in more than one way (at the same time) in the graphical user interface 400. FIG. 4 shows the candidate providers ranked in two ways concurrently in the graphical user interface 400, but other examples can include more, less, or different orderings of the candidate providers in the graphical user interface 400. The server 104 may determine these various orderings and provide them to the computing device for display in the graphical user interface 400.

In some examples, the graphical user interface 400 can also specify a top candidate in another frame 404. The top candidate can be the best candidate provider relative to the other candidate providers on the list. The server 104 can determine the top provider based on one or more parameters, such as the ranks of the provider in multiple ordered lists of the candidate providers. In one such example, the server 104 can generate multiple ordered lists of the candidate providers. The server 104 can add together the ranks of a particular provider in all of the lists, to determine an overall rank value for the candidate provider across all of the lists. The server 104 can repeat this process for all of the candidate providers and compare their overall rank values to one another to identify the top provider. In examples in which lower rank values are deemed better than higher rank values (e.g., a rank of one may be better than a rank of five), then the top provider may be the prover with the lowest overall rank value. If two providers have the same overall rank value, then one of the two providers may be selected randomly or based on other factors. The server 104 can then transmit the top provider (e.g., as a recommendation) to the computing device for display in the graphical user interface 400.

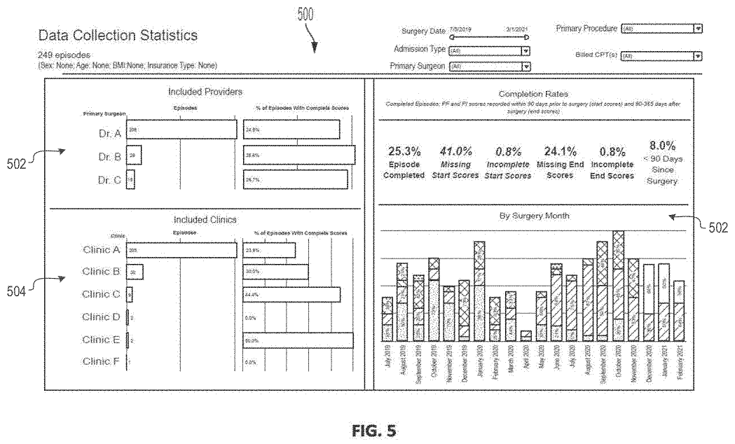

It will be appreciated that the graphical user interface 400 shown in FIG. 4 is intended to be illustrative and non-limiting. Other examples may include more graphical features, less graphical features, or different graphical features than are shown in FIG. 4. For instance, in some examples the graphical user interface 400 can include additional information, such as one or more additional pages (e.g., dashboards) that are accessible to a user by selecting one or more buttons or tabs. Some users may have greater access to information in the pages than other users, so that access to certain pages or page modules is restricted to designated users. The designated users may be users with certain roles or jobs. Examples of these additional pages are shown in FIGS. 5-14 and described below. These additional pages can be generated by the server 104 and transmitted to the computing device for display in the graphical user interface 400.

FIG. 5 shows a page 500 of a graphical user interface that describes data collection statistics for treatment episodes and completion rates associated with various service providers across various clinics. As shown, the page 500 can include a frame 502 with a list of providers and the number of episodes they have treated. The page 500 can also include another frame 504 with statistics about locations at which the episodes were treated. Yet another frame 506 may include similar information broken down by timeframe. This page 500 may help a user (e.g., an administrator) monitor treatment results and identify possible areas of improvement.

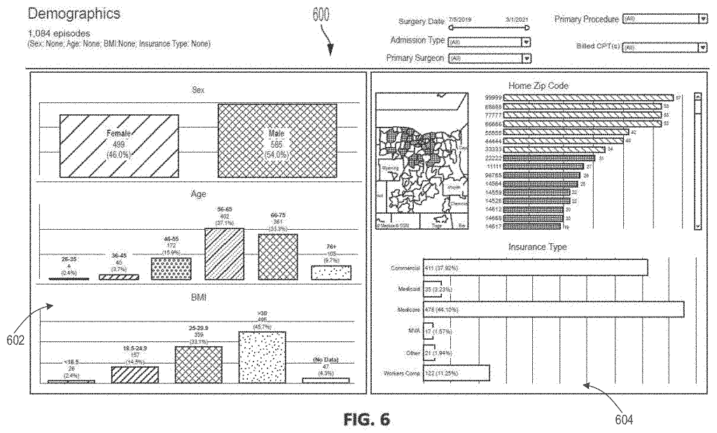

FIG. 6 shows a page 600 of the graphical user interface that describes DCs and insurance information relating to the treatment episodes. For example, the left frame 602 categorizes the episode information by DC groups, while the right frame 604 categorizes the episode information into other groups. The episode information may additionally or alternatively be categorized in other ways, as desired by an administrator. The administrator may be able to select, in the graphical user interface, how they would like to view the episode information and the graphical user interface can responsively generate the corresponding breakdown. This page 600 may also help a user monitor treatment results and identify possible areas of improvement.

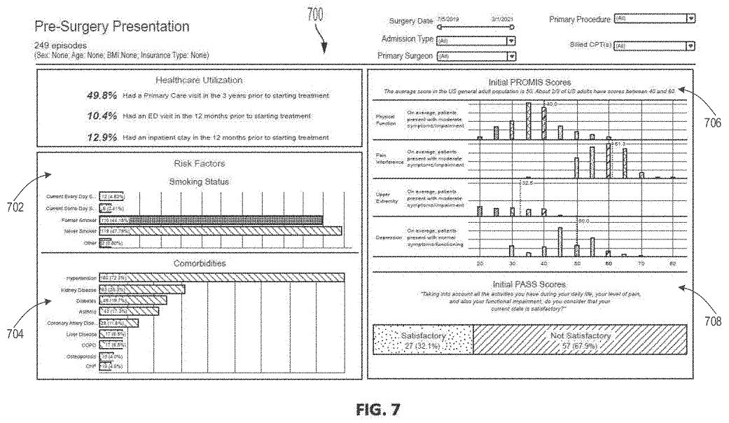

FIG. 7 shows a page 700 of the graphical user interface describing various risk factors 702, comorbidities 704, initial PROMIS scores 706, and initial PASS scores 708 related to patients, among other things. PASS stands for Patient-Acceptable Symptom State, and is one type of measure of an individual's percept of the acuity of their symptoms/condition. The page 700 can present background information about the patients (e.g., prior to engaging in treatment) to understand how their backgrounds may have influenced treatment results. A user may select which background information to view and the graphical user interface may responsively present the selected information.

While some examples described herein involve PROMIS and PASS scores, this is intended to be illustrative and non-limiting. It will be appreciated that any other similar type of measure (e.g., indicating patient-reported outcomes) may be used additionally or alternatively to such scores, for example in relation to any of the graphical user interfaces described herein.

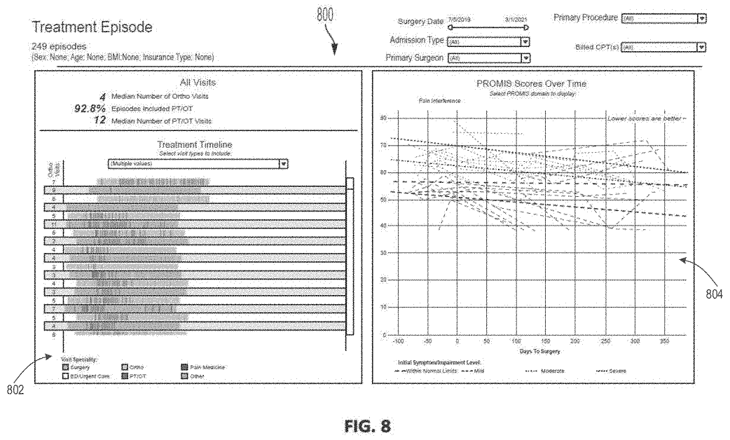

FIG. 8 shows a page 800 of the graphical user interface that describes statistics about treatment episodes, treatment timelines, and variations in PROMIS scores over time. The page 800 includes a left frame 802 with a treatment timeline and a right frame 804 with a graph showing PROMIS scores over time. The graph can show a patient's pain level within a timeframe before and after surgery. This page 800 may help a user understand the typical treatment cycle for an individual patent or a cluster of patients.

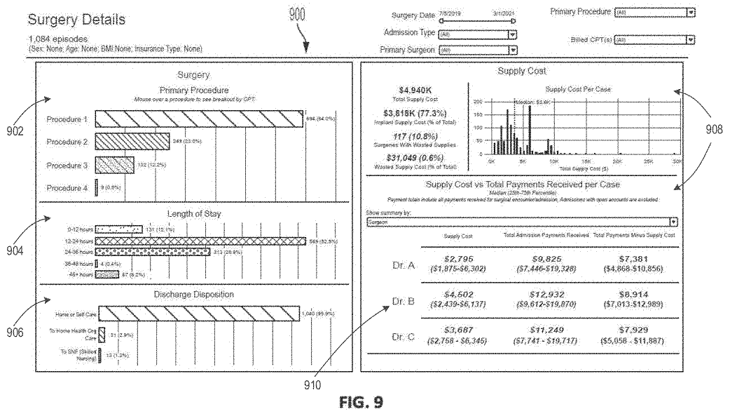

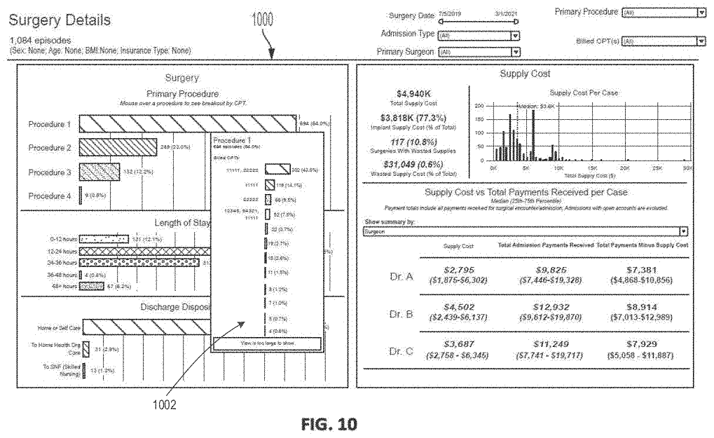

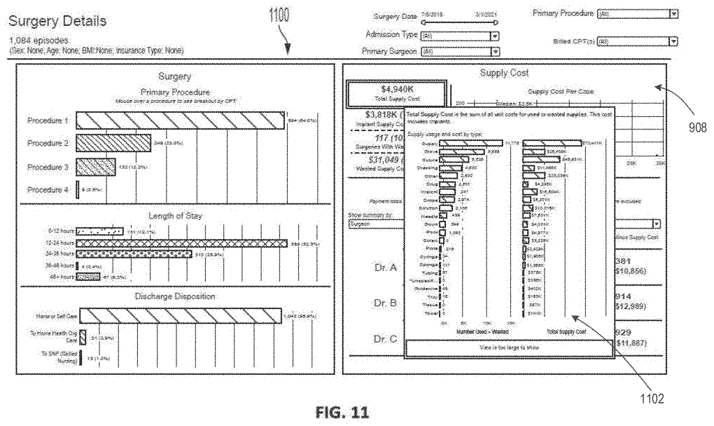

FIG. 9 shows a page 900 of the graphical user interface that describes information about surgeries such as the primary procedure performed 902, the length of stay at the recovery center 904, a discharge disposition 906, and associated overhead information 908 (e.g., supply cost vs. payments received per case). The overhead information may be broken down by individual practitioners 910. In some examples, a user can select or hover their mouse over one of the listed procedures to view additional information about the procedure. The additional information may be shown in a frame or a popup window, for example as shown in FIG. 10. Referring now to FIG. 10, shown is the page 900 of FIG. 9 when a user hovers a mouse over or clicks on one of the bars corresponding to the procedures. As shown, the graphical user interface can generate a popup 1002 with information about charges. In this example, the information includes Current Procedural Terminology (CPT) codes and corresponding percentages in which they were applied to the selected procedure. In some examples, the user may also select or hover their mouse over a metric in the overhead information 908 to view additional information related to that metric. The additional information may be shown in a frame or a popup window, for example as shown in FIG. 11. Referring now to FIG. 11, shown is the page 900 of FIG. 9 when a user hovers a mouse over or clicks on the box for total supply cost. As shown, the graphical user interface can generate a popup 1102 with a breakdown of usage and expenditures. This may help the user gain a better understanding over the overhead breakdown associated with the procedures.

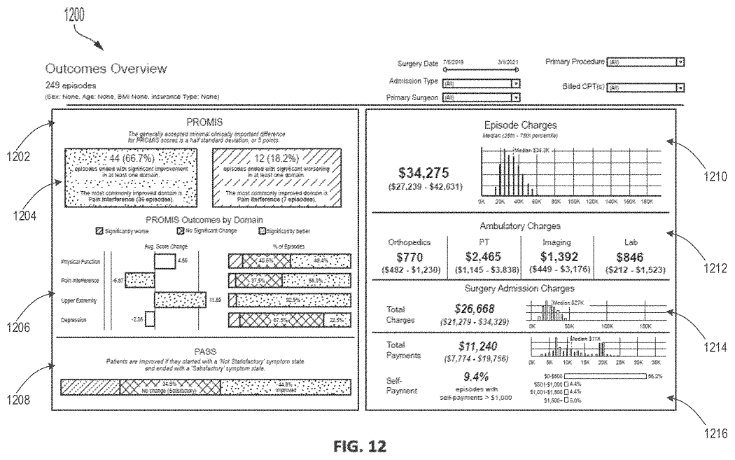

FIG. 12 shows a page 1200 of the graphical user interface describing episode outcomes and charges. In particular, the left frame 1202 includes graphs indicating episode outcomes. The episode outcomes are categorized by PROMIS scores 1204, domain 1206, and PASS changes 1208. The right frame 1210 includes graphs and metrics about charges and payments 1216 relating to the episodes, such as ambulatory 1212 and surgery admission 1214 charges.

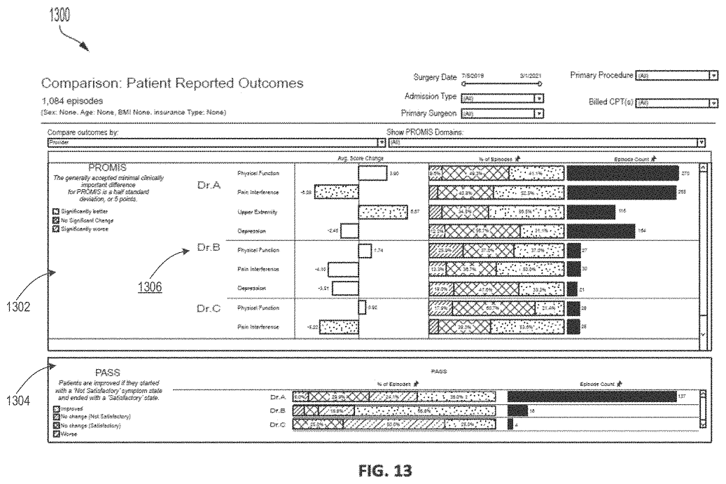

FIG. 13 shows a page 1300 of the graphical user interface that describes comparisons of outcomes between treatment providers 1306 (e.g., doctors). The page 1300 includes a top frame 1302 and a bottom frame 1304. The top frame 1302 includes graphs with PROMIS and episode metrics related to each provider 1306. The bottom frame 1304 includes graphs showing PASS scores achieved by different providers.

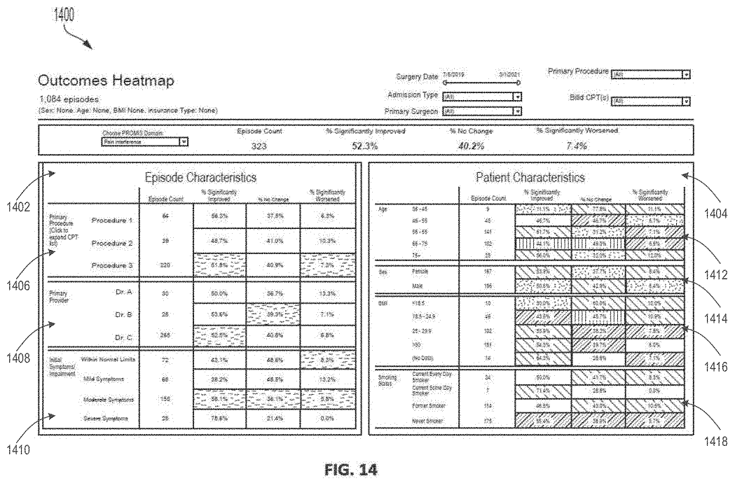

FIG. 14 shows a page 1400 of the graphical user interface that includes heat maps (HMs) 1406-1418 of treatment outcomes based on episode characteristics and patient characteristics. In particular, the page 1400 includes a left frame 1402 with HMs 1406-1410 related to episode characteristics. The page 1400 also includes a right frame 1404 with HMs 1412-1418 related to patient characteristics. The HMs may help the user visualize these characteristics in a more intuitive manner.

It will be appreciated that the examples shown in FIGS. 5-14 are intended to be illustrative and non-limiting. Other examples may include more graphical features, less graphical features, different graphical features, or different arrangement of graphical features than are shown in each of FIGS. 5-14. For example, some of the graphical interface pages show frames split horizontally, but in other examples the frames may be split vertically (or vice-versa). The order and way in which a user may navigate between those graphical interface pages may also be different in other examples.

The above description of certain examples, including illustrated examples, has been presented only for the purpose of illustration and description and is not intended to be exhaustive or to limit the disclosure to the precise forms disclosed. Modifications, adaptations, and uses thereof will be apparent without departing from the scope of the disclosure. For instance, any examples described herein can be combined with any other examples to yield further examples.

1. A method for searching one or more databases for data values based on a search query, the method comprising:
receiving, by a processing device, the search query from a computing device through an interactive graphical user interface, the search query indicating an attribute and a condition of a target client, the attribute being different from the condition;
in response to receiving the search query, determining, by the processing device, a plurality of similarity values corresponding to other clients described in the one or more databases based on the attribute, each similarity value of the plurality of similarity values indicating a respective similarity between the target client and one of the other clients;
selecting, by the processing device, a subset of the other clients by comparing the plurality of similarity values to a predefined similarity threshold;
filtering, by the processing device, the subset to determine affected clients treated for the condition;
identifying, by the processing device, candidate providers that treated the affected clients for the condition based on relationships between the affected clients and the candidate providers in the one or more databases;
generating, by the processing device, an ordered list of the candidate providers based on treatment metrics associated with the candidate providers; and
providing, by the processing device, a response to the computing device for causing the interactive graphical user interface to display the ordered list of the candidate providers.

2. The method of claim 1, further comprising:
receiving first data from a first remote system over one or more networks, wherein the first data specifies attributes of the other clients;
receiving second data from a second remote system over the one or more networks, wherein the second data specifies attributes of the candidate providers;
receiving third data from a third remote system over the one or more networks, wherein the third data specifies the relationships between the affected clients and the candidate providers; and
including the first data, the second data, and the relationships in the one or more databases.

3. The method of claim 1, further comprising determining the plurality of similarity values by executing a model.

4. The method of claim 3, wherein the model includes a neural network or a classifier.

5. The method of claim 1, further comprising:
receiving, from the computing device and through the interactive graphical user interface, a user input indicating a candidate provider selected from among the ordered list of the candidate providers; and
in response to receiving the user input, arranging for the candidate provider to treat the condition of the target client.

6. The method of claim 1, wherein the attribute of the target client includes a socioeconomic characteristic of the target client, a medical history of the target client, or a preference of the target client.

7. The method of claim 1, wherein the condition includes a neurological condition, an orthopedic condition, a vascular condition, an oncological condition, an ocular condition, a urological condition, or a gastrological condition.

8. The method of claim 1, further comprising determining the ordered list of the candidate providers based on attributes of the candidate providers.

9. The method of claim 8, wherein the attributes of the candidate providers include skill levels of the candidate providers, training levels of the candidate providers, areas of expertise or specialization of the candidate providers, communication styles of the candidate providers, preferences of the candidate providers, or engagement levels of the candidate providers.

10. A non-transitory computer-readable medium comprising program code that is executable by a processing device for causing the processing device to:
receive a search query from a computing device through an interactive graphical user interface, the search query indicating an attribute and a condition of a target client, the attribute being different from the condition;
in response to receiving the search query, determine a plurality of similarity values corresponding to other clients described in one or more databases based on the attribute, each similarity value of the plurality of similarity values indicating a respective similarity between the target client and one of the other clients;
select a subset of the other clients by comparing the plurality of similarity values to a predefined similarity threshold;
filter the subset to determine affected clients treated for the condition;
identify candidate providers that treated the affected clients for the condition based on relationships between the affected clients and the candidate providers in the one or more databases;
generate an ordered list of the candidate providers based on treatment metrics associated with the candidate providers; and
provide a response to the computing device for causing the interactive graphical user interface to display the ordered list of the candidate providers.

11. The non-transitory computer-readable medium of claim 10, further comprising program code that is executable by the processing device for causing the processing device to:
receive first data from a first remote system over one or more networks, wherein the first data specifies attributes of the other clients;
receive second data from a second remote system over the one or more networks, wherein the second data specifies attributes of the candidate providers;
receive third data from a third remote system over the one or more networks, wherein the third data specifies the relationships between the affected clients and the candidate providers; and
include the first data, the second data, and the relationships in the one or more databases.

12. The non-transitory computer-readable medium of claim 10, further comprising program code that is executable by the processing device for causing the processing device to determine the plurality of similarity values by executing a model.

13. The non-transitory computer-readable medium of claim 12, wherein the model includes a neural network or a classifier.

14. The non-transitory computer-readable medium of claim 10, further comprising program code that is executable by the processing device for causing the processing device to:
receive, from the computing device and through the interactive graphical user interface, a user input indicating a candidate provider selected from among the ordered list of the candidate providers; and
in response to receiving the user input, arrange for the candidate provider to treat the condition of the target client.

15. The non-transitory computer-readable medium of claim 10, wherein the attribute of the target client includes a socioeconomic characteristic of the target client, a medical history of the target client, or a preference of the target client.

16. The non-transitory computer-readable medium of claim 10, wherein the condition includes a neurological condition, an orthopedic condition, a vascular condition, an oncological condition, an ocular condition, a urological condition, or a gastrological condition.

17. The non-transitory computer-readable medium of claim 10, further comprising program code that is executable by the processing device for causing the processing device to determine the ordered list of the candidate providers based on attributes of the candidate providers.

18. The non-transitory computer-readable medium of claim 17, wherein the attributes of the candidate providers include skill levels of the candidate providers, training levels of the candidate providers, areas of expertise or specialization of the candidate providers.

19. The non-transitory computer-readable medium of claim 17, wherein the attributes of the candidate providers include communication styles of the candidate providers, preferences of the candidate providers, or engagement levels of the candidate providers

20. A system comprising:
one or more databases;
a processing device communicatively coupled to the one or more databases; and
a memory device comprising instructions that are executable by the processing device for causing the processing device to:
receive a search query from a computing device through an interactive graphical user interface, the search query indicating an attribute and a condition of a target client, the attribute being different from the condition;
in response to receiving the search query, determine a plurality of similarity values corresponding to other clients described in the one or more databases based on the attribute, each similarity value of the plurality of similarity values indicating a respective similarity between the target client and one of the other clients;
select a subset of the other clients by comparing the plurality of similarity values to a predefined similarity threshold;
filter the subset to determine affected clients treated for the condition;
identify candidate providers that treated the affected clients for the condition based on relationships between the affected clients and the candidate providers in the one or more databases;
generate an ordered list of the candidate providers based on treatment metrics associated with the candidate providers; and
provide a response to the computing device for causing the interactive graphical user interface to display the ordered list of the candidate providers.
